(19) United States
(12) Patent Application Publication
(10) Pub. No.: US 2026/0165859 A1
(43) Pub. Date: Jun. 18, 2026

(54) BIODEGRADABLE METAL STENT

(71) Applicant: BOSTON SCIENTIFIC SCIMED, INC., Maple Grove, MN (US)

(72) Inventors: Francini Zuniga Luna, Boston, MA (US); James Scutti, Norwell, MA (US)

(73) Assignee: BOSTON SCIENTIFIC SCIMED, INC., Maple Grove, MN (US)

(21) Appl. No.: 19/422,333

(22) Filed: Dec. 16, 2025

Related U.S. Application Data
(60) Provisional application No. 63/735,277, filed on Dec. 17, 2024.

(51) Int. Cl. A61F 2/90

(52) U.S. Cl. CPC A61F 2/90; A61F 2230/0069; A61F 2240/001

(57) ABSTRACT

A biodegradable stent is disclosed. The stent includes a tubular member formed of a plurality of interwoven filaments. The filaments comprise a magnesium alloy, preferably a WE22 magnesium alloy. The magnesium alloy may consist essentially of 1.9-2.1 wt. % Yttrium, 1.4-1.6 wt. % Neodymium, 0.4-0.6 wt. % Zinc, and the balance Magnesium. The filaments may have an ultimate tensile strength of 60 to 80 KSI and/or an elongation at break in the range of 2% to 20%, preferably 2% to 10%.

### CROSS REFERENCE TO RELATED APPLICATIONS

The present application claims the benefit of U.S. Provisional Patent Application Ser. No. 63/735,277, filed Dec. 17, 2024, the disclosure of which is incorporated herein by reference.

### TECHNICAL FIELD

The present disclosure pertains to medical devices, and methods for manufacturing medical devices. More particularly, the present disclosure pertains to biodegradable medical stents, and methods for manufacturing and using such devices.

### BACKGROUND

Stents are utilized in a variety of medical procedures and situations, and accordingly, their structure and function are well known. A stent is generally a tubular prosthesis that is introduced via a catheter into a body lumen. The stent is introduced into the body lumen with a generally reduced diameter and subsequently expanded to the diameter of the body lumen. In its expanded configuration, the stent may support and reinforce the wall of the body lumen while maintaining the body lumen in an open, unobstructed condition.

In some instances, utilizing a medical stent to treat a target site may only require temporary placement of the stent. For example, in some instances it may be beneficial to implant a stent at a target site for a limited period of time, after which it may be desirable to remove the stent (for example, after completion of the treatment). However, it can be appreciated that trauma, such as tearing or similar damage to the wall of the body lumen may occur if the stent is forcibly removed from the body lumen. Accordingly, in some instances it may be desirable to design the stent to biodegrade after the prescribed treatment period. Examples described herein disclose a stent designed to biodegrade after a prescribed treatment period, eliminating the need for subsequent intervention to remove the stent.

### SUMMARY

One example disclosed herein is a biodegradable stent. The stent includes a tubular member having a first end, a second end, and a lumen extending between the first and second ends. The tubular member is formed of a plurality of interwoven filaments. The filaments are formed of a magnesium alloy consisting essentially of: 1.9-2.1 wt. % Yttrium, 1.4-1.6 wt. % Neodymium, 0.4-0.6 wt. % Zinc, and the balance Magnesium.

In addition, or alternatively, to any example disclosed herein, the filaments have an ultimate tensile strength of at least 60 KSI.

In addition, or alternatively, to any example disclosed herein, the filaments have an ultimate tensile strength of 60 to 80 KSI.

In addition, or alternatively, to any example disclosed herein, the filaments have an elongation at break in the range of 2% to 20%.

In addition, or alternatively, to any example disclosed herein, the filaments have an elongation at break in the range of 2% to 10%.

In addition, or alternatively, to any example disclosed herein, the filaments have a diameter in the range of 0.005 inches to 0.020 inches.

In addition, or alternatively, to any example disclosed herein, the filaments comprise a WE22 magnesium alloy.

In addition, or alternatively, to any example disclosed herein, the filaments have been subjected to a heat treatment at 300° C. for 5-15 minutes.

In addition, or alternatively, to any example disclosed herein, the filaments have been subjected to 40% to 75% cold working.

Another example is a biodegradable stent. The stent includes a braided tubular member having a first end, a second end, and a lumen extending between the first and second ends. The braided tubular member is formed of a plurality of filaments interwoven in a one-over-one-under braiding pattern. The filaments comprise a WE22 magnesium alloy. The filaments have been subjected to a heat treatment at 300° C. for 5-15 minutes prior to forming the braided tubular member.

In addition, or alternatively, to any example disclosed herein, the filaments have been subjected to cold working prior to heat treatment.

In addition, or alternatively, to any example disclosed herein, the filaments have been subjected to 40% to 75% cold working.

In addition, or alternatively, to any example disclosed herein, the filaments have an ultimate tensile strength of 60 KSI to 80 KSI.

In addition, or alternatively, to any example disclosed herein, the filaments have an elongation at break in the range of 2% to 10%.

In addition, or alternatively, to any example disclosed herein, the filaments have one of: a diameter of 0.005 inches, an ultimate tensile strength of about 70-80 KSI, and an elongation at break of 6-10%; a diameter of 0.010 inches, an ultimate tensile strength of about 60-65 KSI, and an elongation at break of 6-7%; or a diameter of 0.015 inches an ultimate tensile strength of about 65-70 KSI, and an elongation at break of 2-6%.

In addition, or alternatively, to any example disclosed herein, the WE22 magnesium alloy consists essentially of: about 2.0 wt. % Yttrium, about 1.5 wt. % Neodymium, about 0.5 wt. % Zinc, and the balance Magnesium.

Another example is a method of forming a biodegradable stent. The method includes braiding a plurality of filaments on a mandrel to form a braided tubular member. The filaments are formed of a magnesium alloy. The method further includes applying a tension force to the filaments during braiding, welding free ends of the filaments together, and heat treating the braided tubular member at 300° C. for 5-15 minutes.

In addition, or alternatively, to any example disclosed herein, the magnesium alloy consists essentially of: about 2.0 wt. % Yttrium, about 1.5 wt. % Neodymium, about 0.5 wt. % Zinc, and the balance Magnesium.

In addition, or alternatively, to any example disclosed herein, the tension force is between 0.5-1.5 lbf.

In addition, or alternatively, to any example disclosed herein, the filaments have an ultimate tensile strength of 60 to 80 KSI.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an exemplary stent.

FIG. 2 is a side view of a portion of a braiding machine manufacturing a stent.

FIGS. 3-5 are charts illustrating change in weight of stent samples over time in a test environment.

FIG. 6 is a chart illustrating change in weight of surface damaged stent samples over time in a test environment.

FIGS. 7 and 8 are charts illustrating pH change in stent samples over time in a test environment.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

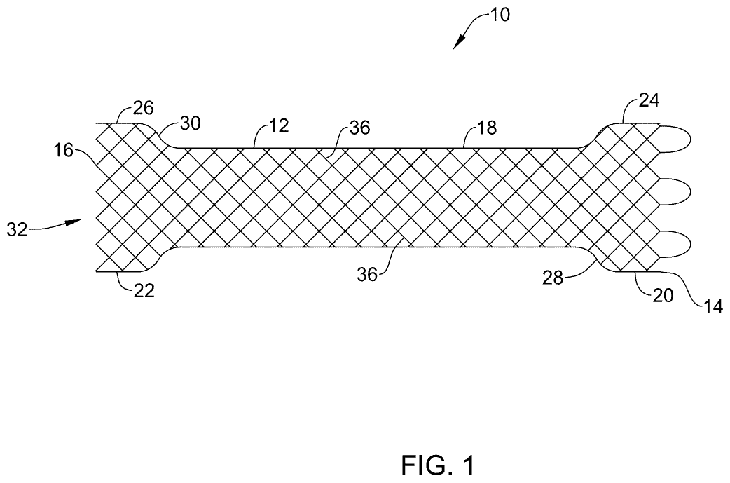

FIG. 1 illustrates a side view of an illustrative endoluminal stent 10. In some instances, the stent 10 may be formed from an elongated tubular member 12. While the stent 10 is described as generally tubular, it is contemplated that the stent 10 may take any cross-sectional shape desired. The stent 10 may have a first, or proximal, end 14, a second, or distal, end 16, and an intermediate region 18 disposed between the first end 14 and the second end 16. The stent 10 may include a lumen 32 extending from a first opening adjacent the first end 14 to a second opening adjacent to the second end 16 to allow for the passage of food, fluids, etc.

The stent 10 may be radially expandable from a first radially collapsed configuration (not explicitly shown) to a second radially expanded configuration. The stent 10 may be structured to extend across a stricture and to apply a radially outward pressure to the stricture in a body lumen to open the body lumen and allow for the passage of foods, fluids, air, etc.

The stent 10 may have a woven structure or scaffold, fabricated from a one or more, or a plurality of interwoven filaments 36. In some embodiments, the stent 10 may be braided with a single filament. In other embodiments, the stent 10 may be braided with a plurality of filaments, as is found, for example, in the WallFlex®, WALLSTENT®, and Polyflex® stents, made and distributed by Boston Scientific, Corporation. In another embodiment, the stent 10 may be knitted from a single filament, such as the Ultraflex™ stents made by Boston Scientific, Corporation, or knitted from a plurality of filaments. In some instances, an inner and/or outer surface of the stent 10 may be entirely, substantially or partially, covered with a polymeric covering or coating. For example, a covering or coating which may help reduce food impaction and/or tumor or tissue ingrowth.

In some instances, in the expanded configuration, the stent 10 may include a first end region 20 and a second end region 22. In some embodiments, the first end region 20 and the second end region 22 may include retention features or anti-migration flared regions 24, 26 positioned adjacent to the first end 14 and the second end 16 of the stent 10. The anti-migration flared regions 24, 26 may be configured to engage an interior portion the walls of the body lumen. In some embodiments, the retention features, or flared regions 24, 26 may have a larger diameter than the intermediate region 18 of the stent 10 to prevent the stent 10 from migrating once placed in the body lumen. It is contemplated that the transition 28, 30 from the cross-sectional area of the intermediate region 18 to the retention features or flared regions 24, 26 may be gradual, sloped, or occur in an abrupt step-wise manner, as desired.

In some embodiments, the first anti-migration flared region 24 may have a first outer diameter and the second anti-migration flared region 26 may have a second outer diameter. In some instances, the first and second outer diameters may be approximately the same, while in other instances, the first and second outer diameters may be different. In some embodiments, the stent 10 may include only one or none of the anti-migration flared regions 24, 26. For example, the first end region 20 may include an anti-migration flare 24 while the second end region 22 may have an outer diameter similar to the intermediate region 18. It is further contemplated that the second end region 22 may include an anti-migration flare 26 while the first end region 20 may have an outer diameter similar to an outer diameter of the intermediate region 18. In some embodiments, the stent 10 may have a uniform outer diameter from the first end 14 to the second end 16. In some embodiments, the outer diameter of the intermediate region 18 may be in the range of 15 to 25 millimeters. The outer diameter of the anti-migration flares 24, 26 may be in the range of 20 to 30 millimeters. It is contemplated that the outer diameter of the stent 10 may be varied to suit the desired application.

It is contemplated that the filament(s) 36 forming the stent 10 can be made from a biodegradable material, such as a biodegradable metal alloy, and particularly a biodegradable magnesium alloy. Consideration should be taken to select the biodegradable material. The biodegradable material must not only provide the stent 10 with sufficient mechanical support to provide a radially outward force on the body lumen to open the body lumen, but also interact appropriately with the immune system of the patient.

Degradation of biodegradable metals occurs through an electrochemical corrosion process. Once implanted and exposed to physiological conditions, magnesium alloys undergo anodic dissolution to form magnesium ions (Mg²⁺) and release electrons, which are consumed in cathodic reactions involving water. This produces hydrogen gas and hydroxide ions, leading to the formation of magnesium hydroxide (Mg(OH)₂). The degradation process can be represented by the following reactions:

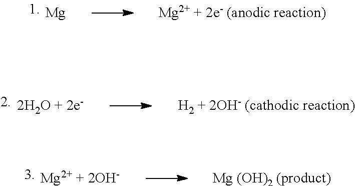

As the degradation proceeds, the magnesium ions that are released at the treatment site create a localized alkaline environment. This alkalization is believed to encourage calcium-phosphate-based apatite deposits that form on the surface of the filament 36, and these apatite deposits could further enhance biocompatibility. However, controlling the degradation rate of the filaments 36 is important as rapid corrosion can compromise mechanical integrity of the stent 10 and produce excessive hydrogen gas, causing complications such as the generation of gas pockets.

Furthermore, upon implantation of the stent 10, proteins may rapidly adsorb onto the surface of the filaments 36 formed of the biodegradable metal alloy, triggering immune responses that can either promote healing or lead to adverse effects. The initial immune response may involve the activation of the complement and clotting cascades, followed by the recruitment of neutrophils and macrophages. These immune cells may attempt to degrade the filaments 36 of the stent 10, and release signaling molecules, such as cytokines, that influence downstream pathways, including myeloid differentiation primary response protein 88 (MyD880, inflammasome, and JAK-STAT signaling. While this inflammatory phase is desirable for tissue repair, uncontrolled or prolonged inflammation can result in fibrosis and stent encapsulation.

Magnesium-based biomaterials have shown promise in modulating immune responses. It is believed that degradation products of magnesium, particularly magnesium ions, exhibit anti-inflammatory effects that support the polarization of macrophages toward the M2 phenotype, which is associated with tissue repair and regeneration. This shift away from the pro-inflammatory M1 phenotype not only reduces fibrosis but also fosters a more favorable environment for healing, highlighting the potential of magnesium-based biomaterials for use in stent construction. The gradual and controlled degradation of magnesium alloys may minimize chronic immune activation typically associated with permanent stents.

One magnesium alloy that provides the stent 10 with sufficient structural support within the body lumen upon initial implantation, while providing an appropriate rate of degradable to prevent premature collapse of the body lumen or prolonged presence in the body lumen is WE22: Mg2Y-1.5Nd-0.5Zn, a magnesium alloy provided by Fort Wayne Medals, Fort Wayne, IN, having the following composition.

| Element | Mg | Y | Nd | Zn |
|---|---|---|---|---|
| Wt. % | Balance | 2 | 1.5 | 0.5 |

In some instances the WE22 magnesium alloy may consist essentially of about 2 wt. % Yttrium (Y), about 1.5 wt. % Neodymium (Nd), 0.5 wt. % Zinc (Zn), and the balance (e.g., about 96 wt. %) Magnesium (Mg). In some instances the WE22 magnesium alloy may consist essentially of 2.01 wt. % Yttrium (Y), 1.46 wt. % Neodymium (Nd), 0.48 wt. % Zinc (Zn), and the balance (e.g., about 96 wt. %) Magnesium (Mg). In some instances, the magnesium alloy may consist essentially of 1.9-2.1 wt. % Yttrium (Y), 1.4 to 1.6 wt. % Neodymium (Nd), 0.4-0.6 wt. % Zinc (Zn), and the balance (e.g., 96.3-95.7 wt. %) Magnesium (Mg).

The filaments 36 may be cold worked and/or heat treated prior to being braided into the stent 10. For instance, the filaments 36 may be subjected to a 40%, 55%, or 75% cold work. Additionally or alternatively, the filaments 36 may be heat treated, such as heat treated in a fluidized bath of alumina sand at 300° C. for 5 to 15 minutes. The following data was collected based on heat treatment of filaments 36 in a fluidized bath of alumina sand at 300° C. for 5 minutes and heat treatment of filaments 36 in a fluidized bath of alumina sand at 300° C. for 15 minutes. Precipitation hardening, followed by ambient air cooling was compared to non-heat-treated filaments. Also, a combination of cold work drawing and precipitation hardening of the wires was tested.

| | | | | | Ultimate | Elongation |
|---|---|---|---|---|---|---|
| | Diameter | Cold Work | Heating | Heating | Tensile | at Break |
| Sample | (in) | Condition | Temp (° C.) | Time (mins) | Strength (KSI) | (%) |
| 1 | 0.015 | 55% | 300 | 5 | 70.264 | 4.56 |
| 3 | 0.015 | 55% | 300 | 5 | 70.392 | 2.26 |
| 4 | 0.015 | 55% | 300 | 15 | 66.477 | 6.42 |
| 5 | 0.015 | 55% | 300 | 15 | 65.762 | 5.78 |
| 6 | 0.015 | 55% | 300 | 15 | 65.762 | 5.78 |
| 8 | 0.015 | 55% | As received | As received | 44.295 | 3.32 |
| 9 | 0.015 | 55% | As received | As received | 54.928 | 2.34 |
| 10 | 0.015 | 40% | 300 | 5 | 60.446 | 7.36 |
| 11 | 0.015 | 40% | 300 | 5 | 60.396 | 7.40 |
| 12 | 0.015 | 40% | 300 | 5 | 60.408 | 6.80 |
| 13 | 0.015 | 40% | 300 | 15 | 59.040 | 7.56 |
| 14 | 0.015 | 40% | 300 | 15 | 59.680 | 2.06 |
| 15 | 0.015 | 40% | As received | As received | 52.541 | 4.60 |
| 16 | 0.015 | 40% | As received | As received | 53.850 | 4.32 |
| 17 | 0.015 | 40% | As received | As received | 54.023 | 6.90 |
| 18 | 0.010 | 55% | 300 | 5 | 63.963 | 7.18 |
| 19 | 0.010 | 55% | 300 | 5 | 63.971 | 5.96 |
| 20 | 0.010 | 55% | 300 | 5 | 64.048 | 6.16 |
| 21 | 0.010 | 55% | 300 | 15 | 61.443 | 7.12 |
| 22 | 0.010 | 55% | 300 | 15 | 61.882 | 7.10 |
| 23 | 0.010 | 55% | As received | As received | 60.302 | 6.34 |
| 24 | 0.010 | 55% | As received | As received | 60.972 | 7.40 |
| 25 | 0.010 | 75% | 300 | 5 | 50.182 | 16.76 |
| 26 | 0.010 | 75% | 300 | 5 | 50.174 | 14.74 |
| 27 | 0.010 | 75% | 300 | 15 | 47.076 | 17.82 |
| 28 | 0.010 | 75% | 300 | 15 | 46.896 | 22.84 |
| 29 | 0.010 | 75% | 300 | 15 | 45.789 | 8.78 |
| 30 | 0.010 | 75% | As received | As received | 48.059 | 2.74 |
| 31 | 0.010 | 75% | As received | As received | 50.268 | 3.32 |
| 32 | 0.010 | 75% | As received | As received | 50.362 | 2.19 |
| 33 | 0.005 | 55% | 300 | 5 | 77.926 | 9.66 |
| 34 | 0.005 | 55% | 300 | 5 | 77.491 | 6.70 |
| 35 | 0.005 | 55% | 300 | 15 | 73.791 | 8.60 |
| 36 | 0.005 | 55% | 300 | 15 | 74.001 | 8.54 |
| 37 | 0.005 | 55% | As received | As received | 74.083 | 3.06 |
| 38 | 0.005 | 55% | As received | As received | 74.455 | 2.94 |
| 39 | 0.005 | 55% | As received | As received | 61.152 | 1.84 |

Tensile testing, as provided above, demonstrates that for all of the filament diameters a 5-minute precipitation hardening heat treatment increases the ultimate tensile strength (i.e., peak stress) of the filaments 36 and for most of the cases even the elongation at break would increase as well. The 15-minute heat treatment also increases the ultimate tensile strength (i.e., peak stress) when compared to the “as received” (i.e., non-cold-worked, non-heat-treated) filament, but not when compared to the filaments subjected to the 5-minute heat treatment at 300° C. Therefore, it may be desirable to subject the filaments 36 to precipitation hardening at 300° C. for 5 minutes prior to braiding the stent 10.

Thus, the filaments 36 forming the braided stent 10 may be formed of a magnesium alloy, such as the WE22 magnesium alloy noted above, having an ultimate tensile strength (i.e., peak stress) of 60 KSI or more, 65 KSI or more, or 70 KSI or more, and/or an elongation at break in the range of 2 to 20%, 2 to 10%, in the range of 3 to 10%, in the range of 3 to 8%, in the range of 5 to 8%, or in the range of 5 to 10%, for example. In some instances, the filaments 36 may have an ultimate tensile strength of 60 to 80 KSI, or 70 to 80 KSI.

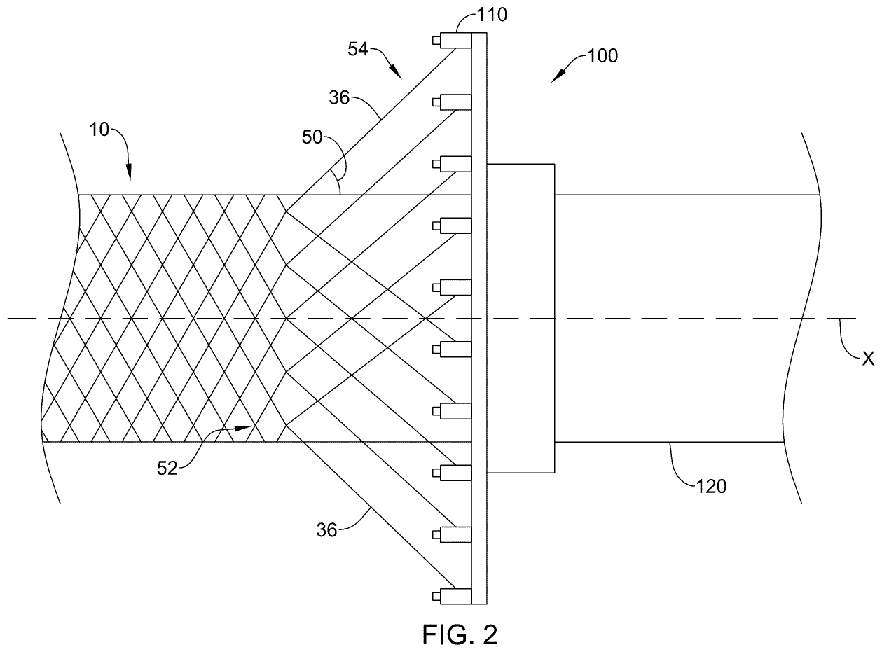

FIG. 2 is a side view illustrating selected aspects of a braiding machine 100 configured to form the stent 10. Some features and/or components of the braiding machine 100, such as motors, controls, safety features, etc. are not shown. However, the braiding machine 100 may include such features and/or components without limitation. In some embodiments, the braiding machine 100 may be a maypole braiding machine. For the purpose of the disclosure, the braiding machine 100 will be described in the context of the maypole braiding machine. However, in some embodiments, the braiding machine may be a rotary braiding machine, or some other type of braiding apparatus.

The braiding machine 100 comprises a plurality of notch gears. In some embodiments, each notch gear may comprise one or more braiding carriers 110 disposed thereon. In some embodiments, each notch gear may comprise a pair of braiding carriers 110 disposed thereon. In some embodiments, the one or more braiding carriers 110 may be disposed on alternating notch gears (e.g., on every other notch gear). The exact arrangement may be dependent upon the number of filaments used to construct the endoprosthesis. A mandrel 120 may extend from and/or through the braiding machine 100. The mandrel 120 may pass through the braiding machine 100 and is movable axially relative to the braiding machine 100. In some instances, a mandrel 120 made of brass providing the mandrel with a smooth, lubricious outer circumferential surface may be used. In some embodiments, the mandrel 120 may be omitted (e.g., the stent 10 may be constructed without the mandrel 120).

The plurality of notch gears may be arranged in a generally circular configuration around the mandrel 120 and/or a central axis X of the braiding machine 100. Each notch gear may be configured to rotate around a center axis thereof, and each notch gear may be configured to rotate in an opposite direction to its neighboring and/or adjacent notch gear(s). As such, the plurality of notch gears may be configured to move in a counter-rotating manner that passes the one or more braiding carriers 110 in a sinusoidal fashion from one notch gear to an adjacent or juxtaposed notch gear, thereby causing the one or more braiding carriers 110 to revolve or move in a circumferential manner around the central axis of the braiding machine 100 around which the plurality of notch gears is arranged. In some embodiments, the central axis may be and/or may be coaxial with a central longitudinal axis X of the mandrel 120. The mandrel 120 is illustrated as having a round configuration (e.g., a circular cross-section), but the mandrel 120 is not necessarily restricted to the round configuration. In some instances, the mandrel 120 may have a square, triangular, etc. configuration or cross-section.

A plurality of filaments 36 may extend from the braiding machine 100 and/or the braiding carriers 110 to the mandrel 120. The braiding machine 100 and/or the braiding carriers 110 may interweave (e.g., braid) the plurality of filaments 36 over and/or around the mandrel 120 to form the tubular scaffold of the stent 10. The circular configuration of the plurality of notch gears and the one or more braiding carriers 110 achieves a generally circular but sinusoidal movement of the one or more braiding carriers 110 to interweave and/or braid the plurality of filaments 36 over and/or around the mandrel 120 to form the stent 10.

In some embodiments, one filament of the plurality of filaments 36 may extend from each braiding carrier of the one or more braiding carriers 110. In some embodiments, more than one filament (e.g., 2 filaments, 3 filaments, 4 filaments, etc.) of the plurality of filaments 36 may extend from each braiding carrier of the one or more braiding carriers 110. In some embodiments, some braiding carriers of the one or more braiding carriers 110 may be devoid of the plurality of filaments 36. As such, the number of filaments used to make the stent 10 may be varied and/or altered while using the same braiding machine 100.

During braiding, the mandrel 120 is moved axially along the central longitudinal axis X in a controlled manner relative to the braiding machine 100. The one or more braiding carriers 110 revolve around the mandrel 120. The plurality of filaments 36 extend from the braiding machine 100 and/or the one or more braiding carriers 110 to the mandrel 120 to form a braid tent 54 having a generally conical configuration, as shown in FIG. 2. The braid tent 54 and/or the plurality of filaments 36 may contact and/or engage with the mandrel 120 at an intersecting angle 50 with an outer surface of the mandrel 120. The intersecting angle 50 is typically between 35 and 65 degrees, and more commonly about 45 to 55 degrees.

The stent 10 produced by the braiding machine 100 of FIG. 2 includes a plurality of filaments 36 interwoven to form a plurality of closed cells defined between adjacent filaments 36. Each closed cell may have corners defined by cross-over points 52 formed by the plurality of filaments 36. In some cases, the mandrel 120 may be configured with securement projections (not shown) extending radially outwardly therefrom the ensure uniform alignment and/or spacing of the plurality of filaments 36 and/or the cross-over points 52, thereby producing the stent 10.

In some embodiments, the plurality of filaments 36 may comprise at least a first filament 36, or a plurality of first filaments 36, extending in a first helical direction around the central longitudinal axis X and at least a second filament 36, or a plurality of second filaments 36, extending in a second helical direction around the central longitudinal axis X different from the first direction. In some embodiments, the stent 10 may comprise multiple filaments 36 (e.g., two, three, four, six, eight, ten, twelve, etc. filaments) extending in the first helical direction around the central longitudinal axis X of the mandrel 120 and/or multiple filaments 36 (e.g., two, three, four, six, eight, ten, twelve, etc. filaments) extending in the second helical direction around the central longitudinal axis X of the mandrel 120. In some embodiments, the multiple filaments 36 extending in the first helical direction around the central longitudinal axis X may be equal in number to the multiple filaments extending in the second helical direction around the central longitudinal axis X. Other configurations are also contemplated. The plurality of filaments 36 may be interwoven and/or braided around the central longitudinal axis X of the mandrel 120 to form a braiding pattern. In some embodiments, the braiding pattern may comprise a one-over and one-under braiding pattern. In some embodiments, the braiding pattern may comprise a two-over and two-under braiding pattern. Other configurations and/or patterns are also contemplated.

In some embodiments, the stent 10 may be manufactured in discrete lengths, wherein the stent 10 is made one at a time. In some embodiments, the stent 10 may be manufactured in a continuous manner (e.g., one long, continuous structure) and cut to desired length(s) at a later time.

The filaments 36 may have any desired diameter. For example, in some instances the filaments 36 may have a diameter in the range of 0.005 inches to 0.020, inches, or in the range of 0.005 inches to 0.015 inches. In some instances, the diameter of the filaments 36 may be 0.005 inches, 0.010 inches, or 0.015 inches, for example. Other diameters of the filaments 36 are also contemplated.

While braiding the stent 10 in the braiding machine 100, constant tension may be applied to the filaments 36 by the braiding carriers 110. For example, the braiding carriers 110 may apply a tension force of 0.5-1.5 lbf to the filaments 36 during the braiding process. The applied tension force may be selected based on the diameter of the filaments 36. Experimental testing has shown that in braiding the stent 10 on a braiding machine as described above, it is desirable to apply a tension force on the filaments 36 by the braiding carriers 110 based on the diameter of the filaments 36 as noted below.

| Filament Diameter (inches) | Tension Force (lbf) |
|---|---|
| 0.005 | 0.5 |
| 0.010 | 0.6 |
| 0.015 | 1 to 1.5 |

After the stent 10 has been braided on the mandrel, or otherwise braided with the braiding machine 100, the free ends of the filaments 36 of the braided stent 10 are then welded together. For example a free end of each filament extending in a first helical direction may be bent at one end of the stent 10 and then welded to a free end of one of the filaments 36 extending in a second helical direction to form loops at the end of the stent 10. For example, the ends of the filaments 36 may be laser welded together. The voltage and size of the weld may be selected based on the size of the filaments 36 being welded. In some instances, the laser welding voltage may be in the range of 200 to 250 Volts, or in the range of 210 to 250 Volts. For example, for filaments 36 having a diameter of 0.005 inches, the laser welding may be performed at 210 Volts and 1 Hz, to form a size 2 spot weld; for filaments 36 having a diameter of 0.010 inches, the laser welding may be performed at 220 Volts and 1 Hz, to form a size 4 spot weld; and for filaments 36 having a diameter of 0.015 inches, the laser welding may be performed at 235 Volts and 1 Hz, to form a size 6 spot weld. The braided tubular member forming the stent 10 may be removed from the braiding machine 100 before or after welding the ends of the filaments 36.

After braiding and welding, the braided stent 10 may be heat-treated. For example, the braided stent 10, while still on the mandrel 120, heat treated at 300° C. for 5 to 15 minutes to shape set the stent 10. In some instances, the braided stent 10, while still on the mandrel 120, may be heat-treated by placing the stent 10 in a white alumina heating bath at 300° C. for 5 minutes to shape set the stent 10.

Sample stents formed of WE22 magnesium alloy filaments in accordance with the braiding process were created for testing. The below table summarizes the stent designs, filament diameters and conditions, and length and diameter of the stent.

| | | Wire | % | Stent | Stent | | |
|---|---|---|---|---|---|---|---|
| Sample | Wires | Diameter | Cold | Diameter | Length | Loop | Manufacturing |
| key | (#) | (inches) | Worked | (mm) | (mm) | Ends | date & Storage |
| A | 10 | 0.010 | 55 | 6 | 100 | Welded | Braided in September |
| B | 7 | 0.010 | 55 | 10 | 80 | Welded | 2024, stored at |
| C | 8 | 0.010 | 55 | 10 | 95 | Open | ambient air with no |
| D | 8 | 0.010 | 55 | 10 | 80 | Welded | temperature control. |
| E | 9 | 0.010 | 75 | 8 | 80 | Welded | Braided in October |
| F | 10 | 0.010 | 75 | 8 | 80 | Welded | 2024, stored in a |
| | | | | | | | sealed bag with no |
| | | | | | | | temperature control. |
| G | 10 | 0.015 | 55 | 10 | 100 | Welded | Braided in June 2024, |
| | | | | | | | stored at ambient air |
| | | | | | | | with no temperature |
| | | | | | | | control |
| H | 10 | 0.005 | 55 | 10 | 100 | Open | Braided in November |
| | | | | | | | 2024, stored in a |
| | | | | | | | sealed bag with no |
| | | | | | | | temperature control. |

WE22 magnesium alloy biodegradable stents formed by the above braiding method were subjected to degradation studies as per ASTM F3160-21. For the stents that were crafted with the WE22 alloy, two different conditioning solutions were used, one representing a bile fluid and the other one representing intestinal/gastric fluid. For the bile a mixture of 0.017 μm CaCl (PN: 38314-7; Sigma Aldrich), 0.12 μm KCl (PN: PX1405-1; EM Science) and 11.28 gm Bovine bile (PN: B-3883; Sigma-Aldrich) was mixed with 200 ml of saline. For the gastric solution the product Simulated Intestinal Fluid TS (PN: 7109-32) by RICCA was used off the shelf.

Each stent was placed in a vial (PN: 60827-207; PYREX) with 150 ml of the corresponding solution. Sample stents A, B, C, E, G, H, and J were placed in a vial of the bile fluid, and sample stents D, F, and I were placed in a vial of the gastric fluid. Bile solution was confirmed to be in a pH range of 6 to 7 after mixing, and the gastric solution has a pH range of 6.7 to 6.9 pH per product specification. Additionally wires were added to individual vials to monitor along with the stents.

All the vials were placed in a Force Digital Bench Top Incubated Shaker (PN: 4628CC; Lab-Line) in a rack and the shaker was maintained at 60° C. and 50 rpm to accelerate the degradation process. The accelerated conditioning was based off the Arrhenius Q₁₀ Accelerated aging model, which shows that a 10° C. increase or decrease in temperature of a homogenous process results in a 2× or ½× change, respectively, per the Arrhenius equation below, wherein;
t is the time required for product to be in an elevated temperature,
T is the actual period being sought or claimed (in days),
b is the elevated temperature being used, and
a is the ambient storage temperature expected for standard product storage

$$t = \frac{T}{{2}^{\frac{( b - a )}{10}}}$$

Considering maximum temperature b equal to 60° C. and ambient temperature equal to 25° C., the table below outlines the time t required in the chamber for each month of actual time of degradation. As a 50-rpm shaking condition is introduced into the accelerated degradation of the sample, it can be considered that this will accelerate the degradation of the samples. Column t (shaking) takes this shaking condition into account.

| | | | | t - shaking |
|---|---|---|---|---|
| | | T (days) | t (days) | (days) |
| 1 | month | 31 | 2.7 | 1.6 |
| 2 | months | 59 | 5.2 | 3.1 |
| 3 | months | 90 | 8.0 | 4.8 |
| 4 | months | 120 | 10.6 | 6.4 |
| 5 | months | 151 | 13.3 | 8.0 |
| 6 | months | 181 | 16.0 | 9.6 |
| 7 | months | 212 | 18.7 | 11.2 |
| 8 | months | 243 | 21.5 | 12.9 |
| 9 | months | 273 | 24.1 | 14.5 |
| 10 | months | 304 | 26.9 | 16.1 |
| 11 | months | 334 | 29.5 | 17.7 |
| 12 | months | 365 | 32.3 | 19.4 |

The accelerated conditioning was conducted for 18 days total, pH and weight measurements were taken up to day 15. For the pH measurements, the Orion Star Benchtop Meter (PN: STARA2115; Thermo Fisher Scientific) was used, 4 buffers were used before each day's measurements to calibrate the meter, between each sample, the meter probe was cleaned with DI water and a non-woven cloth (TX604; Texwipe) to avoid sample contamination. Readings of pH were taken for all stent samples and for the vials with the wire samples, also measurements of the blank solutions were taken.

Weight measurement was collected for the stents. All stents were weighted before the conditioning started. For weight collection. each vial with a stent was emptied in a large dish (PN: 10754-782; VWR) to first inspect the solution contents (stent, crystals, debris) then the stent was removed and gently rinsed with deionized water, airdried and then weighted using a lab scale. After the stent was weighted, a funnel was used to return the solution into the vial and the stent was placed back in the vial. The dish and the funnel were washed and then rinsed with deionized water after each sample to avoid contamination.

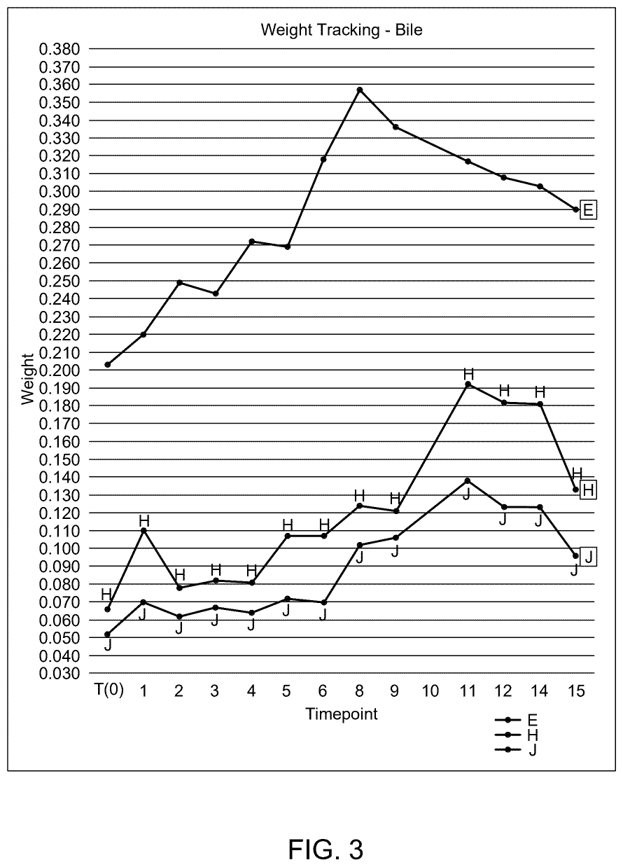

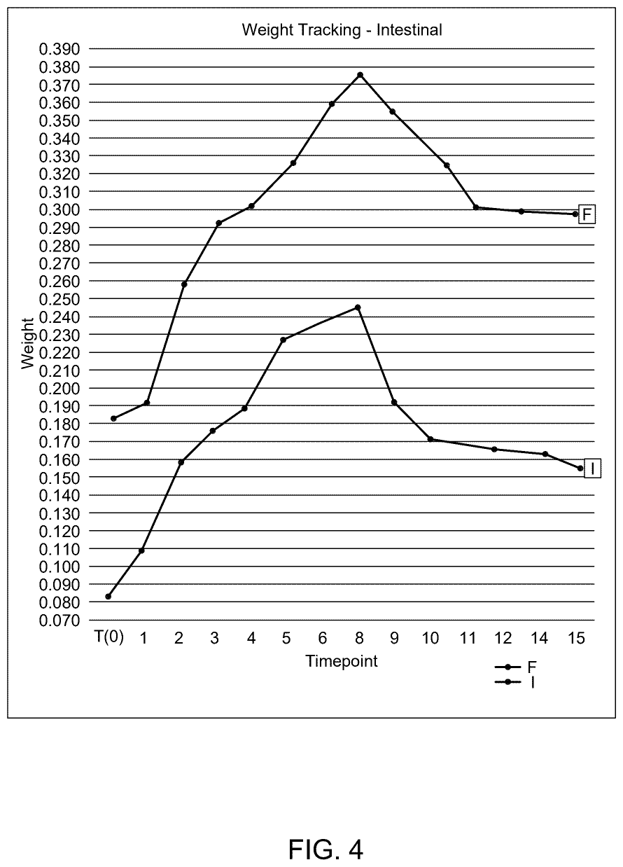

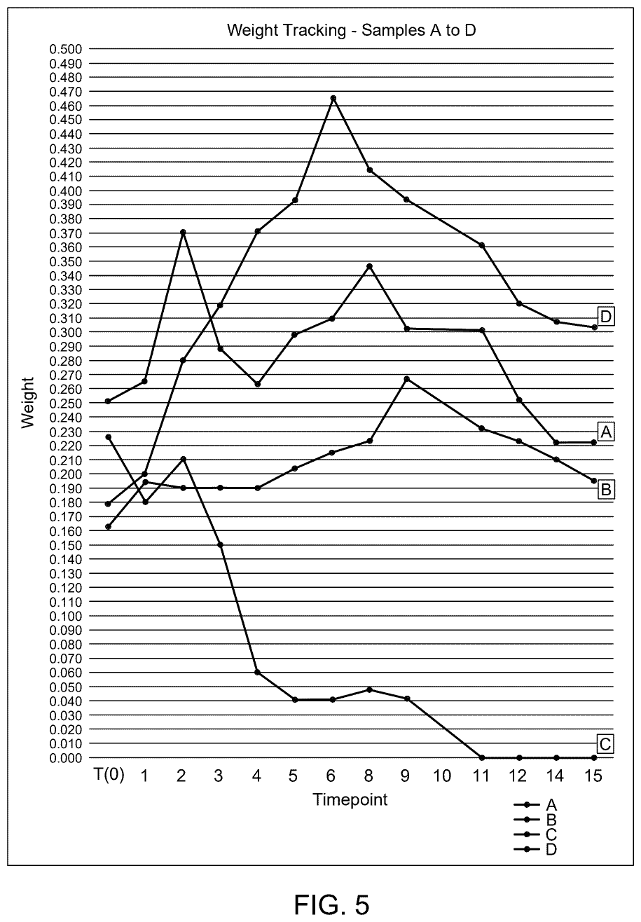

FIG. 3 shows the weight tracking of sample stents E, H and J in the bile fluid. As illustrated in FIG. 3, the percent weight gain for these sample stents was much larger than the percent weight loss. FIG. 4 shows the weight tracking of sample stents F and I in the intestinal fluid. FIG. 5 shows the weight tracking of sample stents A, B, C and D. Reviewing the weight gain and loss of these samples, it can be noted that samples A-D have a ratio of weight gain to weight loss much smaller than samples E, F, H, I and J. This difference is believed to attributed to storage conditions of the samples and damage to the surface of the filaments during the braiding process.

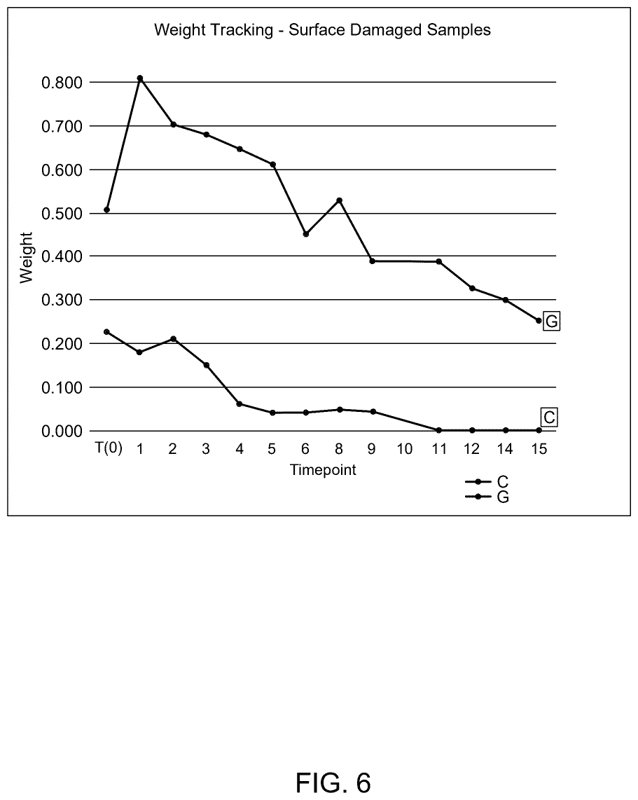

Sample stents C and G experienced surface damage during braiding and/or testing. Sample stent C was damaged during sample monitoring and sample stent G was created with a diamond shaped body mandrel which likely caused surface damage to the wires during braiding, resulting in a more rapid rate of degradation. As shown in FIG. 6, stent G showed a large accumulation of crystals on the surface of the filaments on day one, and stent C showed an accumulation of crystals on the surface of the filaments on day two, after the surface damage occurred.

Weight gain and loss for each sample stent is shown in the table below.

| | A | B | C | D | E | F | G | H | I | J |
|---|---|---|---|---|---|---|---|---|---|---|
| W_{MAX} | 0.370 | 0.267 | 0.226 | 0.465 | 0.357 | 0.376 | 0.810 | 0.192 | 0.245 | 0.138 |
| W_{Initial} | 0.251 | 0.163 | 0.226 | 0.179 | 0.203 | 0.181 | 0.507 | 0.066 | 0.083 | 0.052 |
| W_{MIN} | 0.222 | 0.163 | 0.000 | 0.179 | 0.203 | 0.181 | 0.252 | 0.066 | 0.083 | 0.052 |
| W_{Final} | 0.222 | 0.195 | 0.000 | 0.303 | 0.290 | 0.298 | 0.252 | 0.133 | 0.155 | 0.096 |
| Δ_{GAIN} | 0.119 | 0.104 | 0.000 | 0.286 | 0.154 | 0.195 | 0.303 | 0.126 | 0.162 | 0.086 |
| Δ_{LOSS} | 0.148 | 0.072 | 0.226 | 0.162 | 0.067 | 0.078 | 0.558 | 0.059 | 0.090 | 0.042 |
| GAIN | 47.41 | 63.80 | 0.00 | 159.78 | 75.86 | 107.73 | 59.76 | 190.91 | 195.18 | 165.38 |
| (%) | | | | | | | | | | |
| LOSS | 40.00 | 26.97 | 100 | 34.84 | 18.77 | 20.74 | 68.89 | 30.73 | 36.73 | 30.43 |
| (%) | | | | | | | | | | |

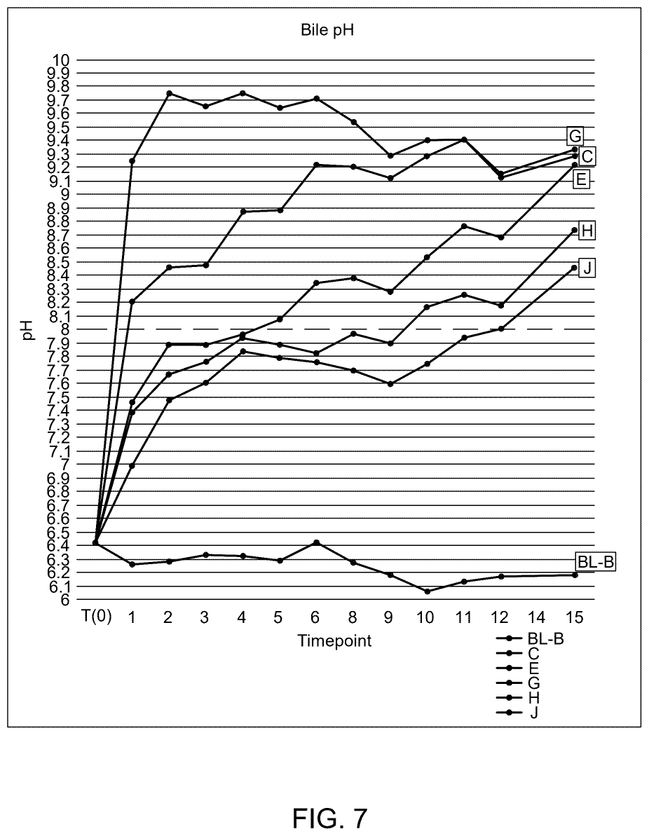

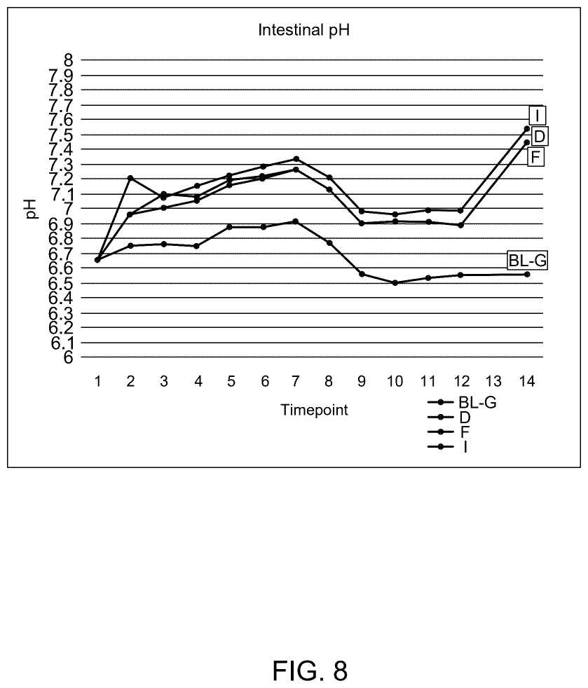

From the pH measurements on the samples that were being conditioned it can be noted that the degradation of the stents is tied to the alkalinity of the environment. As shown in the comparison of FIG. 7 to FIG. 8, it can be noted that the samples in the bile fluid were more susceptible to pH increasing than the samples in the intestinal fluid. Of the samples that were being aged with the bile fluid it seems the degradation (measured by weight loss) is tied to a pH of around 8, show the point at which most of the stents started to lose weight.

For the samples in the bile fluid there seems to be a correlation on crystal proliferation with the rise of the pH of the solution, Sample E as an example of this behavior. It was also observed that there seems to be a limit to the saturation of crystals on the surface of the stent and after this the accumulation of crystals would start to remain on the bottom of the vial with the solution becoming clear.

The pH and weight of all sample stents were monitored until day 15, and the stents were kept in the respective fluid until day 18, at which point the samples were all removed. It was observed that stents B, D, E, F and H maintained their overall shape until the final day. Samples A, C and G broke during the accelerated degradation and samples C and G were mostly dissolved. The samples that survived the degradation process with the design integrity were subjected to manual handling, compressing, stretching and pulling. The samples that were created with the 0.010 inch diameter filaments were able to withstand the manual handling while the samples that were created with the 0.005 inch diameter filaments fractured due to degradation, most likely due to the loss of structural integrity due to the smaller surface area.

Accordingly, it is shown that bioabsorbable stents made from WE22 magnesium alloy may provide temporary structural support to a body lumen and predictable degradation, making them suitable for medical use, such as gastrointestinal and biliary applications.

In some embodiments, the stent 10 may comprise a polymeric covering (not shown) coupled thereto. In some embodiments, the polymeric covering may be fixedly attached to the stent 10. In some embodiments, the polymeric covering may extend along an inner surface of the stent 10. In some embodiments, the polymeric covering may extend along the outer surface of the stent 10. In at least some embodiments, the stent 10 may be embedded within the polymeric covering. Other configurations, including combinations thereof, are also contemplated. Some suitable but non-limiting examples of polymeric materials for the polymeric covering include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyamide (for example, DURETHAN® or CRISTAMID®), elastomeric polyamides, block polyamide/ethers, polyether block amide (PEBA; for example, PEBAX®), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polyurethane silicone copolymers (for example, Elast-Eon® or ChronoSil®), bioabsorbable polymers (for example, poly-l-lactic acid (PLLA), poly lactic-co-glycolic acid (PLGA), etc.), other suitable materials, or mixtures, combinations, copolymers thereof, and the like.

In at least some embodiments, portions or all of the stent 10 and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stent 10 to achieve the same result.

In some embodiments, the stent 10 and/or other components disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A biodegradable stent comprising:
a tubular member having a first end, a second end, and a lumen extending between the first and second ends;
wherein the tubular member comprises a plurality of interwoven filaments;
1.9-2.1 wt. % Yttrium;
1.4-1.6 wt. % Neodymium;
0.4-0.6 wt. % Zinc; and
the balance Magnesium.

2. The biodegradable stent of claim 1, wherein the filaments have an ultimate tensile strength of at least 60 KSI.

3. The biodegradable stent of claim 2, wherein the filaments have an ultimate tensile strength of 60 to 80 KSI.

4. The biodegradable stent of claim 1, wherein the filaments have an elongation at break in the range of 2% to 20%.

5. The biodegradable stent of claim 4, wherein the filaments have an elongation at break in the range of 2% to 10%.

6. The biodegradable stent of claim 1, wherein the filaments have a diameter in the range of 0.005 inches to 0.020 inches.

7. The biodegradable stent of claim 1, wherein the filaments comprise a WE22 magnesium alloy.

8. The biodegradable stent of claim 1, wherein the filaments have been subjected to a heat treatment at 300° C. for 5-15 minutes.

9. The biodegradable stent of claim 1, wherein the filaments have been subjected to 40% to 75% cold working.

10. A biodegradable stent comprising:
a braided tubular member having a first end, a second end, and a lumen extending between the first and second ends;
wherein the braided tubular member comprises a plurality of filaments interwoven in a one-over-one-under braiding pattern;
wherein the filaments comprise a WE22 magnesium alloy; and
wherein the filaments have been subjected to a heat treatment at 300° C. for 5-15 minutes prior to forming the braided tubular member.

11. The biodegradable stent of claim 10, wherein the filaments have been subjected to cold working prior to heat treatment.

12. The biodegradable stent of claim 11, wherein the filaments have been subjected to 40% to 75% cold working.

13. The biodegradable stent of claim 10, wherein the filaments have an ultimate tensile strength of 60 KSI to 80 KSI.

14. The biodegradable stent of claim 10, wherein the filaments have an elongation at break in the range of 2% to 10%.

15. The biodegradable stent of claim 10, wherein the filaments have one of:
a diameter of 0.005 inches, an ultimate tensile strength of about 70-80 KSI, and an elongation at break of 6-10%
a diameter of 0.010 inches, an ultimate tensile strength of about 60-65 KSI, and an elongation at break of 6-7%; or
a diameter of 0.015 inches an ultimate tensile strength of about 65-70 KSI, and an elongation at break of 2-6%.

16. The biodegradable stent of claim 10, wherein the WE22 magnesium alloy consists essentially of:
about 2.0 wt. % Yttrium;
about 1.5 wt. % Neodymium;
about 0.5 wt. % Zinc; and
the balance Magnesium.

17. A method of forming a biodegradable stent comprising:
braiding a plurality of filaments on a mandrel to form a braided tubular member, the filaments formed of a magnesium alloy;
applying a tension force to the filaments during braiding;
welding free ends of the filaments together; and
heat treating the braided tubular member at 300° C. for 5-15 minutes.

18. The method of claim 17, wherein the magnesium alloy consists essentially of:
about 2.0 wt. % Yttrium;
about 1.5 wt. % Neodymium;
about 0.5 wt. % Zinc; and
the balance Magnesium.

19. The method of claim 17, wherein the tension force is between 0.5-1.5 lbf.

20. The method of claim 17, wherein the filaments have an ultimate tensile strength of 60 to 80 KSI.
